(19) United States
(12) Plant Patent Application Publication
(10) Pub. No.: US 2003/0009810 P1
(43) Pub. Date: Jan. 9, 2003

(54) Chrysanthemum plant named 'Swing Time Dark'

(76) Inventor: Mark Roland Boeder, Den Haag (NL)

Correspondence Address: Mark K. Dickson, HOWREY SIMON ARNOLD & WHITE, LLP, 750 Bering Drive, Houston, TX, 77057-2198, US

(21) Appl. No.: 09/900,239

(22) Filed: Jul. 6, 2001

(51) Int. Cl. A01H 5/00

(52) U.S. Cl. PLT/298

(57) ABSTRACT

A new variety of Chrysanthemum plant named ‘Swing Time Dark’ having deep orange red bronze single type flowers and a slightly domed capitulum form and branching habit.

### BACKGROUND OF THE INVENTION

The present invention comprises a new and distinct cultivar of Chrysanthemum botanically known as Dendranthema grandiflora, and referred to by the cultivar name ‘Swing Time Dark.’

‘Swing Time Dark’, identified as 20230-3, was discovered as a natural mutation of a whole plant in a controlled cultivated planting of the variety ‘Swing Time.’ The variety ‘Swing Time Dark’ has been asexually reproduced by vegetative cuttings at Chichester, West Sussex, United Kingdom and the distinguishing characteristics are retained through successive generations of asexual reproduction.

### BRIEF SUMMARY OF THE INVENTION ‘

Swing Time Dark’ is a pot type of Chrysanthemum plant variety having a slightly domed capitulum form and deep orange red bronze single flowers. The age of ‘Swing Time Dark’ in the photo is 60 days from the onset of short days.

### Comparison with Parent

Plant habit of the new Chrysanthemum variety ‘Swing Time Dark’ is similar to the parent cultivar ‘Swing Time.’ In side-by-side comparisons in Chichester, West Sussex, United Kingdom, under commercial practice, plants of the new Chrysanthemum variety ‘Swing Time Dark’ differed from plants of the parent cultivar ‘Swing Time’ in the following characteristics.

1. Flowers of the new variety ‘Swing Time Dark’ are a more deep orange red color than ‘Swing Time.’

### BRIEF DESCRIPTIONS OF ILLUSTRATIONS

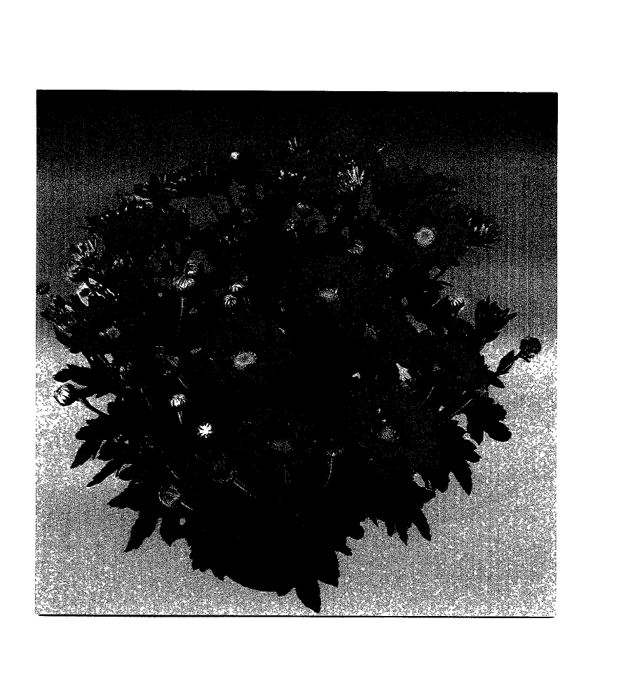

FIGURE 1 is a whole plant view of the new variety ‘Swing Time Dark’ grown in a pot. The colors shown are as true as possible within the usual limits of this kind of illustration.

### DETAILED BOTANICAL DESCRIPTION

The following description of the new Chrysanthemum variety ‘Swing Time Dark’ is of plants grown in a greenhouse in Chichester, West Sussex, United Kingdom in the month of June. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about [e.g., 5,000 to 6,000] foot candles. The plants respond well to the use of growth retardant, such as three B9 treatments at about 2500 ppm. To produce a commercial product the plants may be pinched once. The typical container size for commercial growth is 14 cm. It has been observed that the shelf life of the new variety is about 21 days with a response time of about 7 ½ weeks.

The following description is with respect to a plant produced as a pot Chrysanthemum. In the description of this new Chrysanthemum variety, color values have been taken from The Royal Horticultural Society Colour Chart (R.H.S.C.C.).

### Plant

Plant type: pot

Habit: upright and branching

Height: medium/tall

Width: 32 cm

Branching characteristics: free and spreading

Length of Lateral Branches: 15-20 cm

Number of Breaks from Pinch: 4-6

Stem Color: near 137B

Response Time: near 54 days

Vigor: medium/tall

Shelf Life: near 21 days

Disease (susceptibility or resistance observed): no observations

Growth retardant type and treatment: 3 applications of 2.5 grams/litre at 2 days, 21 days and 28 days after sticking of unrooted cuttings

Propagation:

Type.—vegetative propagation via stem cuttings

Time to rooting.—12-14 days with soil temperatures of 18° C.

Rooting habit.—fine andfibrous

### Foliage

Number of Leaves Per Lateral Branch: 10-14

Compound or single: single

Arrangement of Leaves: alternate

Shape of leaf.—typically 5 lobed, ovate

Size of leaf.—Width (cm): near 4-6 cm Length (cm): near 6-8 cm

Leaf apex.—rounded

Base.—cordate

Attachment.—petioled

Aspect.—slightly undulating

Margin.—palmately lobed

Surface characteristics.—Top: pubescent Bottom: pubescent

Petiole: Color: near 137C Length: near 2 cm

Venation (describe): net, prominent midvein at underside Color: upper side: near 137B underside: near 137D

Color: Mature Leaf, upper side: 137A; under side: near 137D Young Leaf, upper side: near 138A; under side: near 137C

### Flower

Flower type: single

Flower shape: flat and circular

Flowering habit: uniform

Number of blossoms per branch: between 4 and 6

Inflorescence form: pyramidal Depth of fully expanded blossoms: near 2 cm Diameter of fully expanded blossoms: 5-7 cm

Sepals:

Number.—20-25

Color.—near 137B

Length.—0.8-1.2 cm

Width.—0.2-0.3 cm

Texture/appearance.—pubescent Pedicel length is more or less equal over all flowers and between 7 and 8 cm

Length.—7-8 cm

Color.—near 137B

Surface.—pubescent

Habit.—slightly undulating

Strength.—medium

Ray Florets:

Form/shape.—reflexing 1

Texture/appearance.—matte

Number per flower.—34-40

Length.—2.5-4 cm; Width: 0.6-1 cm

Apex.—rounded some slightly dentate

Base.—tapered

Margin.—entire

Disc florets:

Form/shape.—cylindrical

Texture/appearance.—shiny

Number per flower.—160-220

Length.—0.4-0.6 cm; Width <0.1 cm

Diameter of disc.—1-1.6 cm

Fragrance: faint

Flower Bud (at onset of color):

Length.—near 0.8 cm

Diameter.—near 1 cm

Form/shape.—globular

General flower color:

1. Ray florets, upper side.—Immature: near 34A Mature: near 30A Older/Fading: near 30B

2. Ray florets, under side.—Immature: near 34B Mature: near 30B Older/Fading: near 30B

3. Disc florets.—Immature: near 151B Mature: near 7A Older/Fading: near 7A

4. Bud.—near 138B

Flower progression with age: Flowers slightly fading from deep orange red bronze to orange bronze with age.

### Reproductive Organs

Ray Florets: Pistillate

Stigma color.—near 6C

Stigma shape.—forked

Style color.—near 6C

Style length.—near 0.5 cm

Disc Florets: Pistillate

Stigma color.—near 6C

Stigma shape.—forked

Style color.—near 6C

Style length.—near 0.1 cm

Staminate.—

Stamen number.—<5

What is claimed is:

1. A new and distinct variety of Chrysanthemum plant, substantially as described and illustrated herein.
